(19) United States
(12) Patent Application Publication
(10) Pub. No.: US 2014/0353356 A1
(43) Pub. Date: Dec. 4, 2014

(54) Stapler for Use with Distinctively Shaped Staples

(71) Applicant: Victoria Kabluyen, Las Vegas, NV (US)

(72) Inventor: Victoria Kabluyen, Las Vegas, NV (US)

(21) Appl. No.: 13/910,022

(22) Filed: Jun. 4, 2013

(51) Int. Cl. A61B 17/068

(52) U.S. Cl. CPC A61B 17/068
USPC 227/176.1

(57) ABSTRACT

The invention generally pertains to a stapler for applying a distinctively shaped staple to a receiving body, such as a plurality of paper pages. The distinctively shaped staple typically comprises a shaped face top member coupled to a plurality of straight legs. The stapler generally comprises a base plate provided with an anvil for bending the straight legs of the distinctively shaped staple, a staple magazine pivotally connected to the other end of the base plate to be pivotal with respect to the base plate sized and configured to receive and dispense a plurality of distinctively shaped staples, and an actuating lever pivotally connected to the staple magazine and also pivotal with respect to the base plate. The actuating lever is configured to actuate a staple driver to drive a distinctively shaped shaped staple from the staple magazine into the receiving body.

### FIELD OF THE INVENTION

Aspects of embodiments described herein apply to a unique implementation of a stapler, specifically a stapler for passing uniquely shaped staples through paper, tags, and other materials and clenching the staples.

### BACKGROUND OF THE INVENTION

There are many staplers available that close a generally U-shaped staple by bending spaced parts of a central portion of the staple around the surfaces of an anvil so that projecting legs of the staple will engage and join adjacent materials adjacent the anvil, whereupon the anvil can be withdrawn from within the closed, generally rectangular, staple which will then hold the materials together. Such staplers generally include a base plate provided at its free end with an anvil for bending the staple legs, a staple magazine pivotally connected to the other end of the base plate to be pivotal in a plane perpendicular to the base plate, and an actuating lever pivotally connected by means of a separate bearing to the staple magazine to also be pivotal in a plane perpendicular to the base plate to actuate a staple driver to drive the staples from the staple magazine. It is against this background that the following disclosure is presented.

### BRIEF SUMMARY

In one embodiment, a stapler for applying a distinctively shaped staple to a receiving body is provided. Typically, instead of the typical U-shaped design where the staple is constructed of a uniformly sized wire, the distinctively shaped staple has a shaped face coupled to a plurality of straight legs. In one embodiment, the stapler comprises a handle having a staple driver attached adjacent an end of the handle. The staple driver is configured for striking the shaped face of the staple. Continuing, the stapler further comprises a base having an anvil attached adjacent an end of the base. The staple driver is typically aligned with the anvil. The anvil is configured for bending the plurality of straight legs of the distinctively shaped staple. The stapler also has a magazine with a pusher coupled to a spring and rod. The magazine is configured to receive a plurality of distinctively shaped staples. In one embodiment, the magazine has an opening at one end to dispense a single distinctively shaped staple when the staple driver is actuated. In operation, when the handle is actuated, the legs are configured to bend after passing through a receiving body and thereafter attach the distinctively shaped staple to the receiving body.

The foregoing and other features, utilities and advantages of the invention will be apparent from the following more particular description of various embodiments of the invention as illustrated in the accompanying drawings and claims.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates one embodiment of a stapler showing the various components removed from the completed construction, including the stapler base, stapler handle and stapler magazine;

FIG. 2 illustrates one embodiment of a base and anvil;

FIG. 3 shows one embodiment of a handle and staple driver;

FIG. 4 illustrates one embodiment of a magazine;

FIG. 5 illustrates the magazine of FIG. 4 having a spring, metal rod and pusher for placing the distinctively shaped staples into position to be discharged from the stapler;

FIG. 6 illustrates one embodiment of the distinctively shaped staples for use with the stapler of FIG. 1; and

FIG. 7 shows a perspective view of the stapler in operation, showing the pivoting of the handle, magazine and base.

### BRIEF DESCRIPTION

Reference will now be made in detail to embodiments of the invention, examples of which are illustrated in the accompanying drawings. Throughout the following detailed description, the same reference characters refer to the same or similar elements in all figures.

In the following description, numerous specific details are set forth, such as examples of specific shapes, components etc., in order to provide a thorough understanding of the present invention. It will be apparent, however, to one skilled in the art that the present invention may be practiced without these specific details. In other instances, well known components or methods have not been described in detail, but rather in general terms in order to avoid unnecessarily obscuring the present invention. Thus, the specific details set forth are merely exemplary. The specific details may be varied from and still be contemplated to be within the spirit and scope of the present invention.

Reference to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

In one embodiment, the base plate, staple magazine and actuating lever are made of a metallic material, however, one of ordinary skill in the art will appreciate that other materials may be used without departing from the scope and spirit of the present invention. Typically, the three main parts are connected together in pairs by separate, riveted pivot members defining pivot axes.

### Stapler Construction

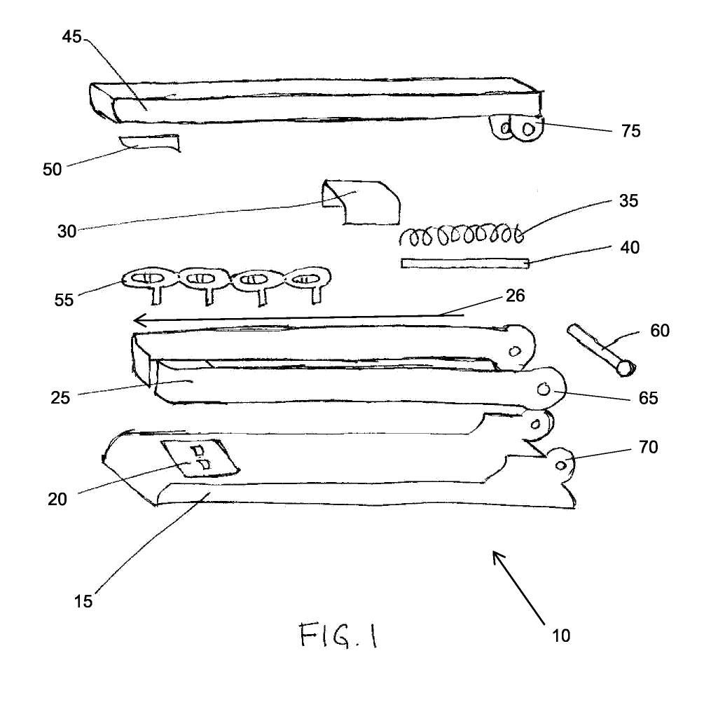

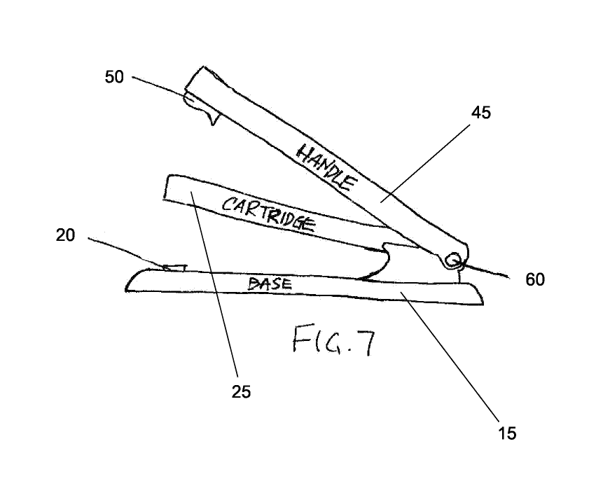

The stapler shown in FIG. 1 essentially includes three parts: the base plate 15; the staple magazine 25; and the actuating lever 45. The staple magazine 25 operates in a manner corresponding to that of known staple magazines, with the exception that it is configured to receive and use a plurality of distinctively shaped staples 55, which are illustrated in FIG. 6 as circular with an opening in the center, but may comprise any number of unique shapes (such as triangle, geometric, company logo to name a few) without departing from the scope and spirit of the present invention. In one embodiment, distinctively shaped staples 55 have a face 56 and a plurality of legs 57, 58. As shown in FIG. 6, the width of the face 56 measured in the direction of 59 is at least twice the width of each of the plurality of staple legs, also measured in the direction of 59. A strip of distinctively shaped staples 55 is disposed within staple magazine 25 to be longitudinally movable in the direction of arrow 26.

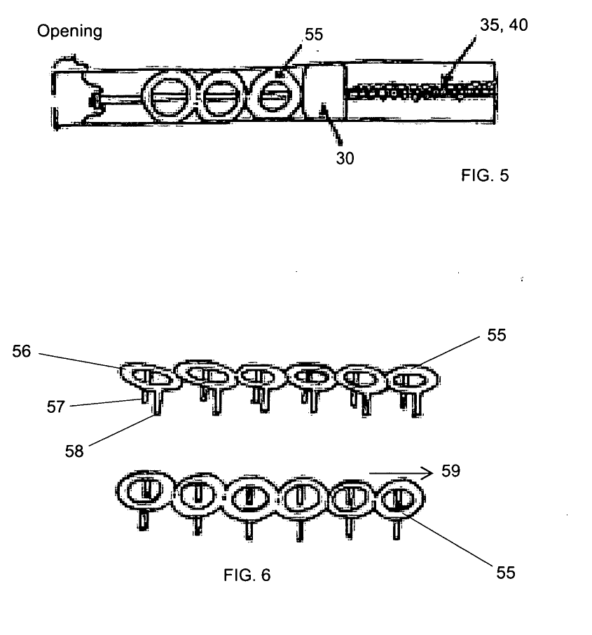

As shown in FIGS. 1, 4 and 5, the staple magazine 25 is provided with a compression spring 35 arranged to urge the distinctively shaped staple strip 55 in the direction of arrow 26, spring 35 being shown in its compressed state in FIG. 5 and in its expanded state in FIG. 1. Compression spring 35 is placed around a guide rod 40 which is fastened within staple magazine 25 to extend in the longitudinal direction 26. Also mounted on guide rod 40 is a pusher or staple guide 30, provided with an opening through which rod 40 passes. Around this opening, pusher 30 presents an abutment surface against which bears the end of spring 35 which faces in the direction of advance 26. The opposite, or rear, end of compression spring is supported at a supporting flange (not shown), which is firmly connected to staple magazine 26 and which also forms a support for guide rod 40. The end of pusher 30 facing in the direction of advance 26 bears against the hindmost staple of a distinctively shaped staple strip 55 and thus presses distinctively shaped staple strip 55 within the guide section of magazine 25 in the direction 26, toward the front discharge end of the latter.

As shown in FIGS. 1 and 3, the actuating lever 45 has a standard, generally box-shaped configuration. In the interior of this box, a staple driver 50 is fastened by a rivet (not shown) to protrude downwardly in a direction approximately perpendicular to the length of lever 45. In order to expel the foremost staple of a distinctively shaped staple strip 55, the staple driver 50 enters between two parallel guide rails, which are fastened to the front end of the magazine part and extend perpendicularly to the length thereof.

When actuating lever 45 is depressed, a terminal portion of staple driver 50 comes to bear against either the staple strip 55 or pusher 30 in staple magazine 25. Upon completion of a stapling operation, a leaf spring (not shown) serves to lift actuating lever 45 away from magazine 25, thereby moving staple driver 50 upwardly so that pusher 30 can again exert a pushing action against the strip of distinctively shaped staples 55.

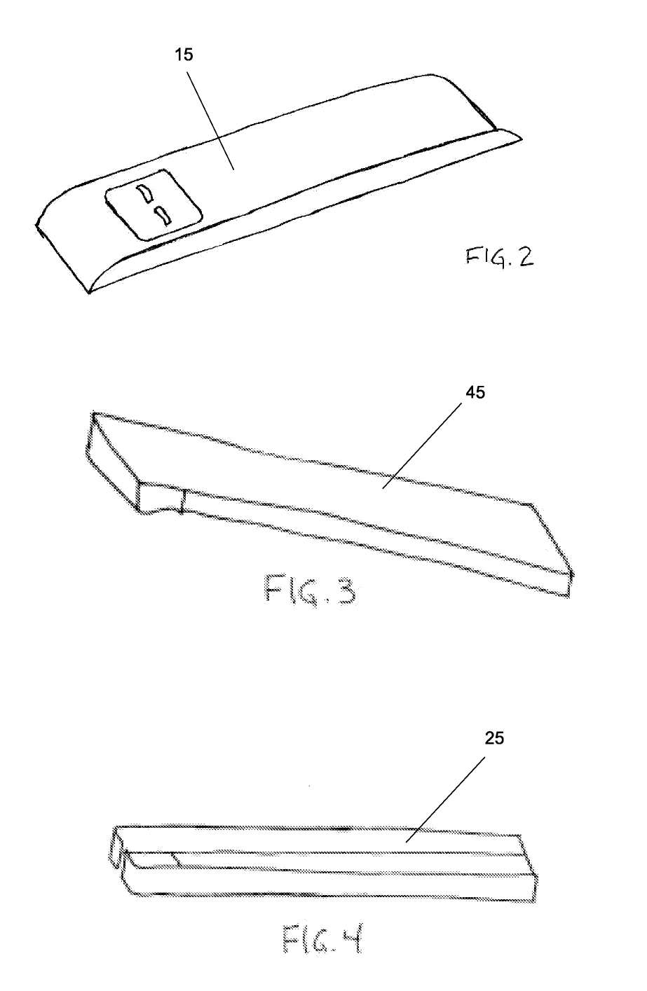

As illustrated in FIGS. 1 and 2, base plate 15 is provided at its front end with an anvil 20 for bending the staple legs, the anvil being formed in a known manner to present two anvil grooves in which the free ends of the legs of a staple passing through the material to be stapled are either spread apart or brought together, in a known manner.

A special pivot connection 65, 70, 75 is provided between staple magazine 25, base plate 15 and handle 45. The longitudinal axis of this pivot connection extends in a known manner perpendicular to the plane of the drawing in FIG. 1. Pivot connection 65, 70, 75 has the form of a plug-in hinge. The axis of this hinge lies just below the plane of the upper surface of base plate 15 and is thus approximately in the plane of the base plate. The pivot connection includes a hinge pin 60.

### Stapler Operation

In the operation of the device, the actuating lever 45 is raised. The raising of the actuating lever 45 exposes the interior of the magazine member 25 (as shown in the top down view in FIG. 5), and loading of the magazine may be accomplished by first retracting the pusher 30. After the pusher has been withdrawn to the rear portion of the staple magazine 25, a strip of distinctively shaped staples 55, cemented together as in the usual manner, may be dropped into the space within the staple magazine 25. The pusher 30 may then be released so as to draw, under the influence of spring 35, the strip of distinctively shaped staples 55 forwardly to the position shown in FIG. 5. The pusher 30 may again be withdrawn to its rear position and another strip of distinctively shaped staples inserted into the space within the staple magazine 25, if desired. The tension exerted by the spring 35 causes the pusher 30 to press the strip of distinctively shaped staples 55 forwardly so that the foremost staple occupies a position close to an opening in staple magazine 25. It will be observed that the driver guide at the actuating lever 45 is forwardly bent so as to provide a cam surface adapted to be engaged in the descending stroke of the staple driver 50. The staple driver 50 thus engages the forward portion of the forward staple without contacting any portion of the next staple.

After the staple magazine 25 has been filled as above described, the actuating lever 45 is closed to the position shown best in FIG. 7, and paper sheets or other receiving members inserted above the anvil 20. In the stapling operation, the actuating lever 45 is pressed sharply downwardly. In this movement, the driver 50 engages the foremost distinctively shaped staple of the plurality of distinctively shaped staples 55 and presses it downwardly. At the same time, the staple magazine 25 moves downwardly. In this position, the distinctively shaped staple is driven home, the end portions piercing the paper sheets and being curved inwardly and clamped by the anvil cam portions 20 of base 15. Upon release of the actuating lever 45, the spring raises staple magazine 25, and spring raises the actuating lever 45. At the same time, spring 35 draws the pusher 30 forwardly so as to bring the next distinctively shaped staple into position against the guides. The apparatus is now ready for the next operation.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A stapler for applying a distinctively shaped staple to a receiving body, said distinctively shaped staple having a shaped face coupled to a plurality of straight legs, said shaped face of said staple having a unique shape and a width that is at least twice the width of each of said plurality of straight legs, said stapler comprising:
a handle having a staple driver attached adjacent an end of said handle, said staple driver is configured for striking said shaped face of said distinctively shaped staple;
a base having an anvil attached adjacent an end of said base, said staple driver aligned with said anvil, said anvil is configured for bending said plurality of straight legs of said distinctively shaped staple; and
a magazine having a pusher coupled to a spring and rod, said magazine configured to receive a plurality of distinctively shaped staples, said magazine having an opening at one end to dispense a single distinctively shaped staple when said staple driver is actuated,
wherein said legs are configured to bend after passing through a receiving body and thereafter attach said staple to said receiving body when said handle is actuated.

2. The stapler of claim 1, wherein said distinctively shaped staple is made of wire.

3. The stapler of claim 1, wherein said distinctively shaped staple further comprises a logo affixed to the shaped face.

4. The stapler of claim 1, wherein said receiving body is a plurality of sheets of paper.

5. A stapler for applying a distinctively shaped staple to a receiving body, said distinctively shaped staple having a shaped face coupled to a plurality of straight legs, said shaped face of said staple having a unique shape and a width that is at least twice the width of each of said plurality of straight legs, said stapler comprising:
a handle having a staple driver attached on a first end and a handle connector for receiving a pin on a second end, said staple driver is configured for striking said shaped face of said distinctively shaped staple;
a base having an anvil attached to a first end and a base connector for receiving a pin on a second end, said staple driver aligned with said anvil, said anvil is configured for bending said plurality of straight legs of said distinctively shaped staple;
a magazine having a pusher coupled to a spring and rod, said magazine configured to receive a plurality of distinctively shaped staples, said magazine having an opening at a first end to dispense a single distinctively shaped staple when said staple driver is actuated, said magazine having a magazine connector for receiving a pin on a second end; and
a pin inserted through said handle connector, said base connector and said magazine connector, wherein said pin pivotally connects said handle, said magazine and said base together and allows said handle and said magazine to pivot relative to said base when said stapler is actuated,
wherein said legs are configured to bend after passing through a receiving body and thereafter attach said distinctively shaped staple to said receiving body when said handle is actuated.

6. The stapler of claim 1, wherein said distinctively shaped staple is made of wire.

7. The stapler of claim 1, wherein said distinctively shaped staple further comprises a logo affixed to the shaped face.

8. The stapler of claim 1, wherein said receiving body is a plurality of sheets of paper.

9. A method of using a stapler for applying a distinctively shaped staple to a receiving body, said distinctively shaped staple having a shaped face coupled to a plurality of straight legs, said shaped face of said staple having a unique shape and a width that is at least twice the width of each of said plurality of straight legs, said method comprising:
providing a stapler having,
a handle having a staple driver attached adjacent an end of said handle, said staple driver is configured for striking said shaped face of said distinctively shaped staple,
a base having an anvil attached adjacent an end of said base, said staple driver aligned with said anvil, said anvil is configured for bending said plurality of straight legs of said distinctively shaped staple, and
a magazine having a pusher coupled to a spring and rod, said magazine configured to receive a plurality of distinctively shaped staples, said magazine having an opening at one end to dispense a single distinctively shaped staple when said staple driver is actuated,
wherein said legs are configured to bend after passing through a receiving body and thereafter attach said staple to said receiving body when said handle is actuated,
providing a plurality of distinctively shaped staples having a shaped face coupled to a plurality of straight legs;
loading said stapler magazine with a plurality of distinctively shaped staples;
inserting a receiving body between said stapler handle and said stapler base, in-line with the opening in said stapler magazine configured for dispensing a distinctively shaped staple;
actuating the stapler by pressing on the stapler handle thereby pushing the staple driver into the shaped face of one of said distinctively shaped staples and forcing said plurality of staple legs of said distinctively shaped staple into said anvil causing said stapler legs to penetrate through said receiving body and bend to secure said distinctively shaped staple to said receiving body.
